(19) United States
(12) Patent Application Publication
(10) Pub. No.: US 2023/0147719 A1
(43) Pub. Date: May 11, 2023

(54) MEDICAL INFORMATION PROCESSING APPARATUS AND MEDICAL INFORMATION PROCESSING SYSTEM

(71) Applicants: CANON MEDICAL SYSTEMS CORPORATION, Tochigi (JP); National Cancer Center, Tokyo (JP)

(72) Inventors: Yuka SHIMOMURA, Nasushiobara (JP); Anri YAMAZAKI, Nasushiobara (JP); Longxun PIAO, Nasushiobara (JP); Minoru NAKATSUGAWA, Yokohama (JP); Naoki MIHARA, Chuo-ku (JP); Masami MUKAI, Chuo-ku (JP); Noriaki NAKAJIMA, Chuo-ku (JP)

(73) Assignees: CANON MEDICAL SYSTEMS CORPORATION, Tochigi (JP); National Cancer Center, Tokyo (JP)

(21) Appl. No.: 18/051,205

(22) Filed: Oct. 31, 2022

(30) Foreign Application Priority Data
Nov. 5, 2021 (JP) 2021-181011

(51) Int. Cl. G16H 10/60; G16H 40/20

(52) U.S. Cl. CPC G16H 10/60; G16H 40/20

(57) ABSTRACT

A medical information processing apparatus according to an embodiment includes a processing circuitry. The processing circuitry acquires operation information of a medical worker for medical treatment information of a target patient or operation information of a medical worker for medical treatment information of a similar patient who is similar to the target patient. Based on the operation information, the processing circuitry calculates the concordance rate between a period for which the medical treatment information of the target patient or the medical treatment information of the similar patient is referred to and the period for which the medical treatment information of the target patient is displayed. The processing circuitry performs display of information about the concordance rate.

### CROSS-REFERENCE TO RELATED APPLICATIONS

This application is based upon and claims the benefit of priority from Japanese Patent Application No. 2021-181011, filed on Nov. 5, 2021; the entire contents of which are incorporated herein by reference.

### FIELD

Embodiments disclosed in the present specification and drawings generally relate to a medical information processing apparatus and a medical information processing system.

### BACKGROUND

In a hospital, for example, when a physician checks the medical treatment information of a target patient, the period for which the medical treatment information of the target patient is displayed is different depending on a disease of the target patient, the purpose of referring to the medical treatment information, and the like. For example, when a physician wants to check the effect of treatment on a target patient, the physician sets a display period so that the physician can compare the past medical treatment information and the current medical treatment information, and in case of emergency, the physician sets the display period so that the physician can check the most recent medical treatment information. Thus, when checking each piece of medical treatment information of the target patient, the physician needs to set the display period for each piece of medical treatment information. However, it takes time and efforts for physicians to set the display period.

### BRIEF DESCRIPTION OF THE DRAWINGS

FIG. 1 is a diagram illustrating an example of a structure of a medical information processing system including an electronic medical record server according to a first embodiment;

FIG. 2 is a flowchart expressing a procedure of a process by the electronic medical record server according to the first embodiment;

FIG. 3A is a flowchart expressing the procedure of the process by the electronic medical record server according to the first embodiment;

FIG. 3B is a flowchart expressing the procedure of the process by the electronic medical record server according to the first embodiment;

FIG. 4 is a diagram for describing the process by the electronic medical record server according to the first embodiment;

FIG. 5 is a diagram for describing the process by the electronic medical record server according to the first embodiment;

FIG. 6 is a diagram for describing the process by the electronic medical record server according to the first embodiment;

FIG. 7 is a diagram for describing the process by the electronic medical record server according to the first embodiment;

FIG. 8 is a diagram for describing the process by the electronic medical record server according to the first embodiment;

FIG. 9 is a diagram for describing the process by the electronic medical record server according to the first embodiment;

FIG. 10 is a diagram for describing a process by an electronic medical record server according to a first modification of the first embodiment;

FIG. 11 is a diagram for describing a process by an electronic medical record server according to a second modification of the first embodiment;

FIG. 12 is a diagram for describing a process by an electronic medical record server according to a third modification of the first embodiment;

FIG. 13 is a diagram for describing a process by an electronic medical record server according to a fourth modification of the first embodiment;

FIG. 14 is a diagram for describing a process by an electronic medical record server according to a fifth modification of the first embodiment;

FIG. 15A is a diagram for describing a process by an electronic medical record server according to a sixth modification of the first embodiment;

FIG. 15B is a diagram for describing the process by the electronic medical record server according to the sixth modification of the first embodiment;

FIG. 16 is a flowchart expressing a procedure of a process by an electronic medical record server according to a second embodiment;

FIG. 17A is a flowchart expressing a procedure of a process by an electronic medical record server according to a third embodiment;

FIG. 17B is a flowchart expressing the procedure of the process by the electronic medical record server according to the third embodiment;

FIG. 18 is a diagram for describing a process of a calculating function by the electronic medical record server according to the third embodiment; and

FIG. 19 is a flowchart expressing a procedure of a process by an electronic medical record server according to a fourth embodiment.

### DETAILED DESCRIPTION

A medical information processing apparatus according to an embodiment includes a processing circuitry. The processing circuitry acquires operation information of a medical worker for medical treatment information of a target patient or operation information of a medical worker for the medical treatment information of a similar patient who is similar to the target patient. Based on the operation information, the processing circuitry calculates the concordance rate between a period for which the medical treatment information of the target patient or the medical treatment information of the similar patient is referred to and the period for which the medical treatment information of the target patient is displayed. The processing circuitry performs display of information about the concordance rate.

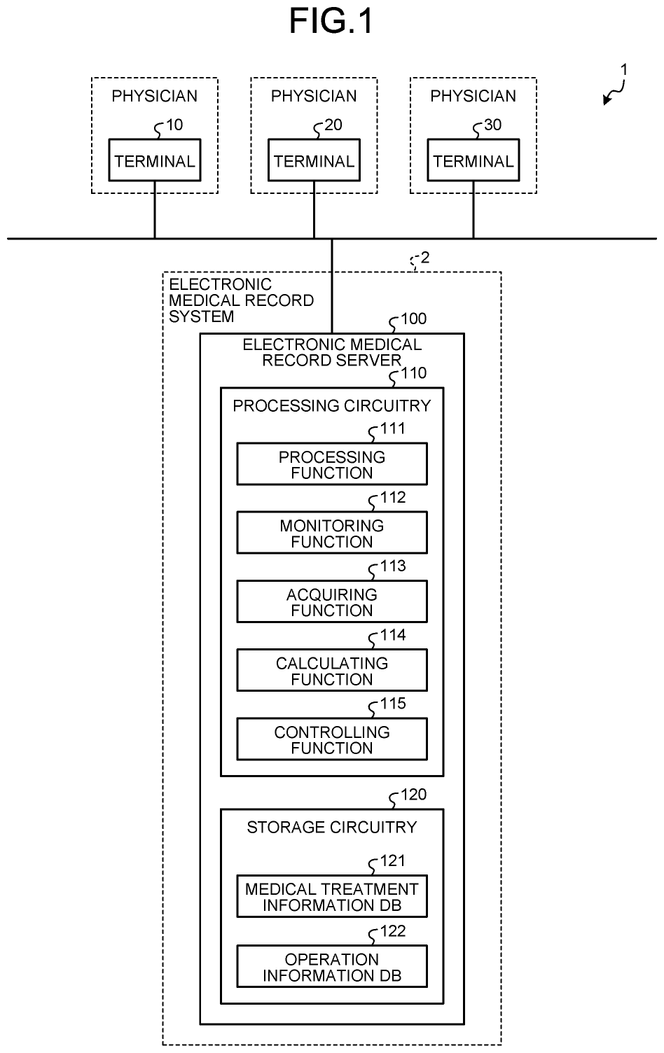

One embodiment of the medical information processing apparatus is described below in detail with reference to the drawings. Note that in the example to be described below, a medical information processing system 1 includes an electronic medical record server incorporating a function of the medical information processing apparatus. In the medical information processing system 1 illustrated in FIG. 1, the respective devices are illustrated one by one but in reality, more devices can be arranged.

### First Embodiment

FIG. 1 is a diagram illustrating one example of a structure of the medical information processing system 1 including an electronic medical record server 100 according to a first embodiment. The medical information processing system 1 illustrated in FIG. 1 includes, for example, an electronic medical record system 2, a radiology information system (RIS), and a picture archiving and communication system (PACS). The electronic medical record system 2 includes, for example, a hospital information system (HIS).

The medical information processing system 1 illustrated in FIG. 1 includes the electronic medical record server 100, terminals 10 to 30, and an HIS server, an RIS server, a medical image diagnosis device, and a PACS server that are not illustrated. The electronic medical record server 100 has a function of the medical information processing apparatus.

The HIS server manages information generated in the hospital. The information generated in the hospital includes patient information, examination order information, and the like. When the examination order information is input from the terminal 10, for example, the HIS server sends the input examination order information and the patient information specified by the examination order information to the RIS server. The RIS server manages examination appointment information related to radiological examination services. The medical image diagnosis device is a device that, for example, performs examinations on the basis of the examination appointment information sent from the RIS server. For example, a clinical technologist performs an examination by, for example, photographing a patient using the medical image diagnosis device, and the medical image diagnosis device generates medical images in performing the examination.

The medical images are, for example, X-ray computed tomography (CT) images, X-ray images, magnetic resonance imaging (MRI) images, nuclear medicine images, and ultrasound images. The medical image diagnosis device converts the generated medical images into a format compliant with the Digital Imaging and Communication in Medicine (DICOM) standard, for example. In other words, the medical image diagnosis device generates medical images to which DICOM tags are added as supplementary information. The supplementary information includes, for example, patient ID, examination ID, device ID, image series ID, etc., and is standardized according to the DICOM standard. The medical image diagnosis device sends the generated medical images to the PACS server. The PACS server, for example, receives the patient information sent from the HIS server and manages the received patient information. For example, the PACS server has a storage circuitry for managing the patient information, receives the medical images sent from the medical image diagnosis device, and stores the received medical images in its own storage circuitry by associating these images with the patient information.

The terminals 10 to 30 are used by the medical workers involved in the medical treatment of the patient. For example, the terminals 10 to 30 are used by physicians. The terminals 10 to 30 include, for example, personal computers (PCs), tablet PCs, personal digital assistants (PDAs), and mobile terminals. The terminals 10 to 30 have a viewer (software) installed therein to display medical images on their own displays.

The electronic medical record server 100 is incorporated into the electronic medical record system 2 together with the HIS server, as described above. The electronic medical record server 100 is communicably connected to the terminals 10 to 30 and the like through a network. The electronic medical record server 100 and the terminals 10 to 30 are connected to an in-hospital local area network (LAN) installed in, for example, a hospital, send information to a predetermined device, and receive information sent from the predetermined device. For example, the electronic medical record server 100 is realized by a computer apparatus such as a workstation or a personal computer.

The electronic medical record server 100 has a processing circuitry 110 and a storage circuitry 120. The electronic medical record server 100 is not limited to the structure described above and the storage circuitry 120 may not be incorporated in the electronic medical record server 100 as long as the electronic medical record server 100 can access the storage circuitry 120 on the network.

The storage circuitry 120 is connected to the processing circuitry 110 and stores various kinds of information therein. Specifically, the storage circuitry 120 stores therein patient information received from each system. For example, the storage circuitry 120 is realized by a semiconductor memory element such as a random-access memory (RAM) or a flash memory, a hard disk, an optical disc, or the like.

The processing circuitry 110 controls components of the electronic medical record server 100. For example, the processing circuitry 110 performs a processing function 111, a monitoring function 112, an acquiring function 113, a calculating function 114, and a controlling function 115. Here, for example, each function performed by the processing function 111, the monitoring function 112, the acquiring function 113, the calculating function 114, and the controlling function 115, which are the components of the processing circuitry 110, is recorded in the storage circuitry 120 in the form of a computer program executable by a computer. The processing circuitry 110 is a processor that reads out each computer program from the storage circuitry 120 and executes the computer program to realize the function corresponding to the computer program. In other words, the processing circuitry 110 that has read out each computer program has each function illustrated in the processing circuitry 110 in FIG. 1.

The processing function 111 performs the function of the electronic medical record system. Specifically, the processing function 111 stores electronic medical records in the storage circuitry 120 that record the patient information such as prescriptions, nursing records, and specimen examinations performed on the patient.

The patient information includes basic information and medical treatment information of the patient. The basic information includes patient ID, name, date of birth, gender, blood type, height, weight, and the like. As the patient ID, identifier information that uniquely identifies the patient is set. The medical treatment information of the patient includes information such as numerical values (measurement values) and medical treatment records, as well as information indicating the date and time of the recording. For example, the medical treatment information of the patient includes prescriptions for medicines by physicians, nursing records by nurses, specimen examinations in the laboratory department, and arrangements for meals during hospitalization. For example, the prescriptions are recorded in the electronic medical records by the physicians, and the nursing records are recorded in the electronic medical records by the nurses.

The monitoring function 112, the acquiring function 113, the calculating function 114, and the controlling function 115 perform the functions of the medical information processing apparatus of the electronic medical record server 100. A display application (computer program) is implemented in the medical information processing apparatus, and the display application can be read out by the terminals 10 to 30. The processes of the monitoring function 112, the acquiring function 113, the calculating function 114, and the controlling function 115 are described later.

The storage circuitry 120 has, as databases (hereinafter simply referred to as “DBs”), a medical treatment information DB 121 and an operation information DB 122. The medical treatment information DB 121, for example, corresponds to a storage circuitry of the HIS server and stores therein the medical treatment information of the patient or the electronic medical record that records the medical treatment information of the patient. The information stored in the operation information DB 122 is described later.

The term “processor” in the above description includes, for example, a central processing unit (CPU), a graphics processing unit (GPU), an application specific integrated circuit (ASIC), a programmable logic device (e.g., simple programmable logic device (SPLD), a complex programmable logic device (CPLD), and a field programmable gate array (FPGA)), and other circuits. If the processor is a CPU, for example, the processor reads out and executes a computer program saved in the storage circuitry 120 to realize the function. On the other hand, if the processor is an ASIC, for example, the computer program is incorporated directly into the circuitry of the processor instead of saving the computer program in the storage circuitry 120. Each processor in this embodiment is not limited to the case where each processor is configured as a single circuit, but may also be configured as a single processor by combining a plurality of independent circuits to realize the functions. Furthermore, a plurality of components in FIG. 1 may be integrated into a single processor to achieve the function.

The overall structure of the medical information processing system 1 including the electronic medical record server 100 according to the present embodiment has been described. With such a structure, the electronic medical record server 100 prevents the oversight of medical treatment information that needs to be checked.

In a hospital, for example, when a physician checks the medical treatment information of a target patient, the period for which the medical treatment information of the target patient is displayed is different depending on a disease of the target patient, the purpose of referring to the medical treatment information, and the like. For example, when a physician wants to check the effect of treatment on a target patient, the physician retrieves each piece of medical treatment information of the target patient, and in order to compare the past medical treatment information and the current medical treatment information of the target patient, sets the period from the date and time of the medical treatment to be compared to the current date and time as the display period. Moreover, in the case of an emergency, the physician sets the most recent period as the display period in order to check the most recent medical treatment information of the target patient. If the physician is young or inexperienced, for example, such a physician may set the long display period.

In addition, the period for which the medical treatment information of the target patient is displayed is different depending on the type of medical treatment information. For example, if the physician wants to check the medical treatment information of vital data, blood test results, or the like of the target patient, or other specimen examinations, the physician sets the period from the date and time of the previous medical treatment to the current date and time as the display period in order to compare the previous medical treatment information and the current medical treatment information of the target patient. In addition, the display period may be set long when the physician wants to check the information about the target patient's tumor or the like.

Thus, when checking each piece of medical treatment information of the target patient, the physician needs to set the display period for each piece of medical treatment information. However, it takes time and efforts for physicians to set the display period.

Therefore, the electronic medical record server 100 in this embodiment performs the following process to prevent the oversight of the medical treatment information that needs to be checked. First, in the electronic medical record server 100 according to this embodiment, the acquiring function 113 acquires the operation information of the medical worker for the medical treatment information of the target patient. Based on the acquired operation information, the calculating function 114 calculates the concordance rate between the period for which the medical treatment information of the target patient is referred to and the period for which the medical treatment information of the target patient is displayed, and the controlling function 115 performs display of information about the concordance rate.

Here, for example, if the acquiring function 113 cannot acquire the operation information of the medical worker for the medical treatment information of the target patient, the acquiring function 113 acquires the operation information of the medical worker for the medical treatment information of a similar patient whose disease and the like are similar to those of the target patient. In this case, based on the acquired operation information, the calculating function 114 calculates the concordance rate between the period for which the medical treatment information of the similar patient is referred to and the period for which the medical treatment information of the target patient is displayed, and the controlling function 115 performs display of information about the concordance rate. As an example, description is made of a case in which the operation information of the medical worker for the medical treatment information of the target patient is acquired.

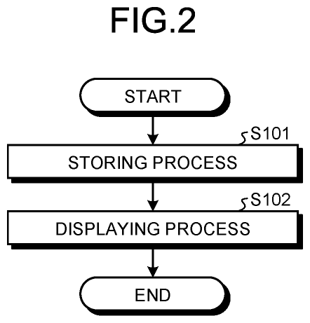

FIG. 2 is a flowchart expressing the procedure of the electronic medical record server 100 according to the first embodiment.

At step S101 in FIG. 2, the processing circuitry 110 calls a computer program corresponding to the monitoring function 112 from the storage circuitry 120 and executes the computer program. At step S101, the monitoring function 112 performs a storing process. For example, the monitoring function 112 monitors the operations of the terminals 10 to 30 of the physicians, who are medical workers, and stores the logs of the operations for the medical treatment information of the patient in the operation information DB 122 as the operation information. Specifically, the monitoring function 112 monitors the operations of a plurality of physicians in the respective hospital departments and stores the contents of the operations by the physicians for the medical treatment information of the patient and the date and time when the operations are performed in the operation information DB 122 as the operation information. Since the monitoring function 112 monitors the physicians' operations (actions), the operation information collected by the monitoring function 112 is also referred to as action data representing the physicians' actions.

Here, for example, the operation information includes the time for which the physician refers to the medical treatment information and the history when the physician accessed the medical treatment information as the contents of the operations by the physician. The time for which the physician refers to the medical treatment information includes click operations and a gazing time by eye tracking. For example, each of the terminals 10 to 30 includes a camera to capture images of a physician operating the terminal, and at step S101, the monitoring function 112 analyzes the images captured by the camera to measure the physician's line of sight (eye tracking) to collect the gazing time by the eye tracking.

At step S102 in FIG. 2, the processing circuitry 110 calls the computer programs corresponding to the acquiring function 113, the calculating function 114, and the controlling function 115 from the storage circuitry 120 and executes the function of each computer program. At step S102, the acquiring function 113, the calculating function 114, and the controlling function 115 execute the displaying process shown below.

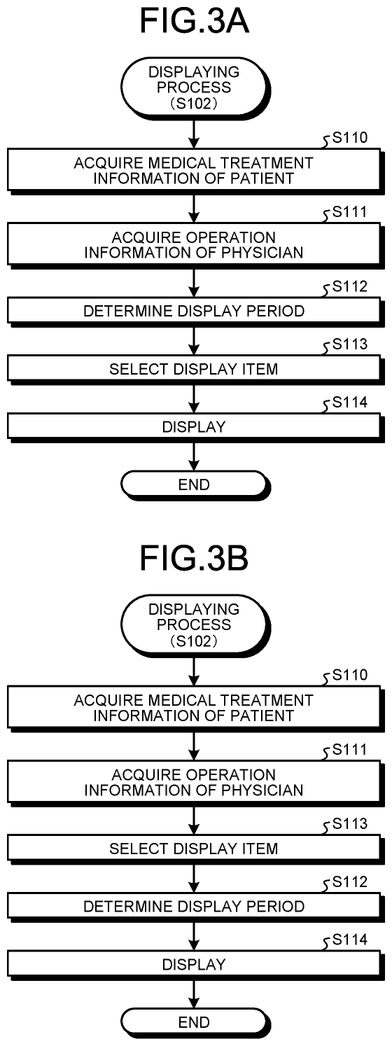

First, the process of the electronic medical record server 100 according to the first embodiment is described with reference to FIG. 3 to FIG. 15B. FIG. 3A and 3B are flowcharts each expressing the procedure of the process by the electronic medical record server 100 according to the first embodiment.

At step S110 in FIG. 3A, the acquiring function 113 of the electronic medical record server 100 acquires the medical treatment information of the target patient registered in the medical treatment information DB 121. For example, when a physician who operates the terminal 10 wants to check the medical treatment information of a target patient, the physician uses the terminal 10 to perform a search using the patient ID, etc. Thus, the acquiring function 113 acquires, by item, the medical treatment information of the target patient up to the current date and time from the medical treatment information DB 121. Here, examples of the item include specimen examination, medication treatment using various medicines, and examinations using medical images.

At step S111 in FIG. 3A, the acquiring function 113 of the electronic medical record server 100 acquires the physician's operation information for the medical treatment information of the target patient acquired by item. The operation information acquired by the acquiring function 113 is the information operated by the physicians in the respective hospital departments. Note that the acquiring function 113 is one example of “acquisition unit”.

At step S112 in FIG. 3A, the calculating function 114 of the electronic medical record server 100 calculates, by item, the concordance rate between the period for which the medical treatment information of the target patient is referred to and the period for which the medical treatment information of the target patient is displayed, on the basis of the operation information. For example, the calculating function 114 determines the period starting from the date and time when the concordance rate is maximum and ending at the current date and time to be the display period. Here, the method of calculating the concordance rate and the method of determining the display period are described later. The calculating function 114 is an example of “calculation unit”.

At step S113 in FIG. 3A, the controlling function 115 of the electronic medical record server 100 selects one or more items to be displayed on the terminal 10 of the physician. The method of selecting the items is described later. Note that the controlling function 115 is an example of “selection unit”.

At step S114 in FIG. 3A, the controlling function 115 of the electronic medical record server 100 causes the terminal 10 of the physician to display information about the concordance rate by item. Specifically, the controlling function 115 causes the terminal 10 of the physician to display, by selected item, the medical treatment information for the determined display period as the information about the concordance rate. The controlling function 115 is an example of “display control unit”.

Here, as illustrated in FIG. 3B, step S112 may be performed after step S113. In this case, after the controlling function 115 selects one or more items to be displayed on the terminal 10 of the physician, the calculating function 114 calculates the concordance rate and determines the display period on the basis of the operation information by selected item.

Next, the process by the electronic medical record server 100 according to the first embodiment will be described with specific examples.

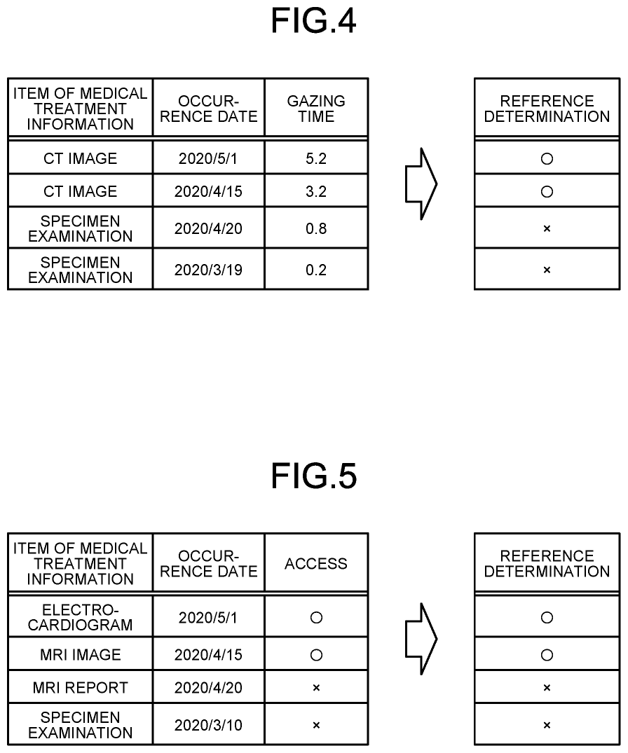

FIG. 4 illustrates an example of the physician's operation information for the medical treatment information of the target patient acquired by item by the acquiring function 113. The operation information includes the time for which the physician refers to the medical treatment information as the contents of the operation by the physician. In FIG. 4, for example, the operation information includes the click operations and the gazing time by eye tracking. In the example in FIG. 4, the operation information includes the items of the medical treatment information “CT image”, “CT image”, “specimen examination”, and “specimen examination”, the respective occurrence dates of the items “2020/5/1”, “2020/4/15”, “2020/4/20”, and “2020/3/19”, and the respective gazing time of the items “5.2 seconds”, “3.2 seconds”, “0.8 seconds”, and “0.2 seconds”. Here, the occurrence date is the date when each of the items “CT image”, “CT image”, “specimen examination”, and “specimen examination” is registered in the medical treatment information DB 121. The gazing time is the time for which the physician refers to each of the items “CT image”, “CT image”, “specimen examination”, and “specimen examination” as the medical treatment information. The calculating function 114 performs a reference determination for the operation information for the gazing time “5.2 seconds”, “3.2 seconds”, “0.8 seconds”, and “0.2 seconds” for the items. For example, if the threshold is set to “2 seconds” and the physician refers to the medical treatment information for “2 seconds” or longer, the calculating function 114 generates a reference determination “◯” for the operation information as the information indicating that the physician has referred to the medical treatment information. On the other hand, if the physician refers to the medical treatment information for less than “2 seconds”, the calculating function 114 generates a reference determination “×” as the information indicating that the physician did not refer to the medical treatment information. In the example illustrated in FIG. 4, the calculating function 114 generates “◯”, “◯”, “×”, and “×” as the reference determination for the operation information respectively for the gazing time of “5.2 seconds,” “3.2 seconds,” “0.8 seconds,” and “0.2 seconds” for the items.

FIG. 5 illustrates an example of the physician's operation information for the medical treatment information of the target patient acquired by item by the acquiring function 113. The operation information includes a history when the physician accessed the medical treatment information as the contents of the operation performed by the physician. In FIG. 5, the operation information is the access history. In the example in FIG. 5, the operation information includes the items of the medical treatment information “electrocardiogram”, “MRI image”, “MRI report”, and “specimen examination”, the occurrence dates of the respective items “2020/5/1”, “2020/4/15”, “2020/4/20”, and “2020/3/10”, and the accesses for the items “◯”, “◯”, “×”, and “×”. Here, the occurrence date is the date and time when each of the items “electrocardiogram”, “MRI image”, “MRI report”, and “specimen examination” is registered in the medical treatment information DB 121. The access is the history when physicians accessed each of the items “electrocardiogram”, “MRI image”, “MRI report”, and “specimen examination” as the medical treatment information. The calculating function 114 performs a reference determination for the operation information of the accesses for the items “◯”, “◯”, “×”, and “×”. For example, if a physician is currently accessing the medical treatment information, the calculating function 114 generates a reference determination “◯” for the operation information as the information indicating that the physician has referred to the medical treatment information. On the other hand, if the physician is not accessing the medical treatment information, the calculating function 114 generates a reference determination “×” for the operation information as the information indicating that the physician is not referring to the medical treatment information. In the example in FIG. 5, the calculating function 114 generates “◯”, “◯”, “×”, and “×” as the reference determination for the operation information of the accesses for the items “◯”, “◯”, “×”, and “×”.

In the example in FIG. 4, the acquiring function 113 can strictly determine whether the physician referred to the medical treatment information by the clicking operation and eye tracking. In the example in FIG. 5, the acquiring function 113 can determine whether the physician referred to the medical treatment information through the existing log collection function such as the click operation.

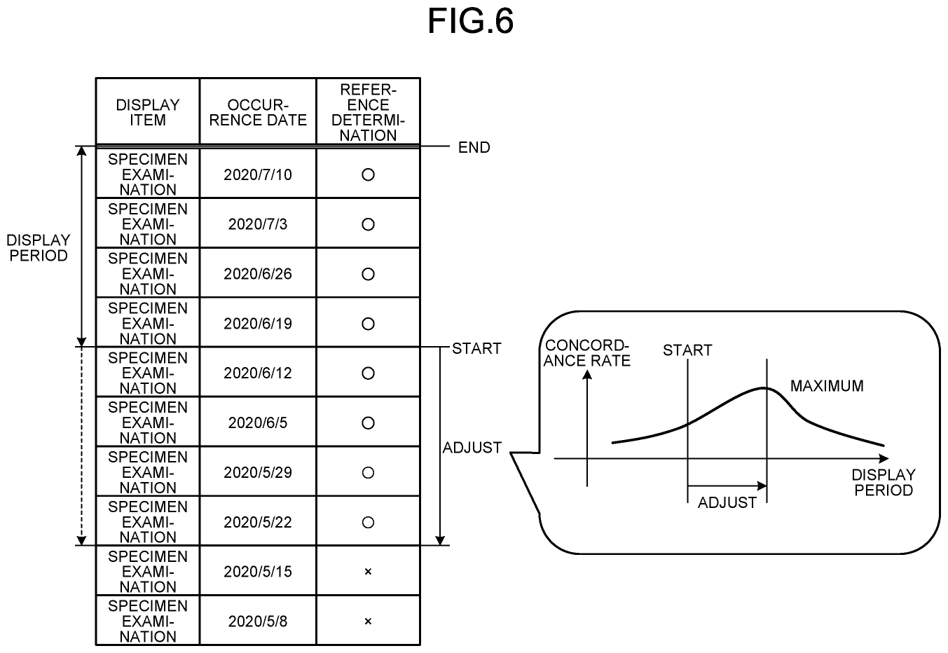

FIG. 6 illustrates another example of the medical treatment information of the target patient acquired by the acquiring function 113 for the item “specimen examination”. In the example in FIG. 6, when the acquiring function 113 acquires the medical treatment information of the target patient for the item “specimen examination”, the medical treatment information from the date “2020/5/8” to the current date “2020/7/10” is expressed among the acquired medical treatment information. Here, the period from the date “2020/5/8” to the current date “2020/7/10” is a display candidate period. Then, the calculating function 114 generates “×”, “×”, “◯”, “◯”, “◯”, “◯”, “◯”, “◯”, “◯”, and “◯” as the reference determination for the operation information, respectively for the occurrence dates of the item “specimen examination”, which are “2020/5/8”, “2020/5/15”, “2020/5/22”, “2020/5/29”, “2020/6/5”, “2020/6/12”, “2020/6/19”, “2020/6/26”, “2020/7/3”, and “2020/7/10”. In this case, in the display candidate period “2020/5/8 to 2020/7/10”, the period for which the reference determination for the operation information is “◯” is from “2020/5/22” to “2020/7/10”. Here, when the calculating function 114 calculates the concordance rate between the period for which the medical treatment information of the target patient is referred to and the period for which the medical treatment information of the target patient is displayed, on the basis of the operation information for the item “specimen examination”, the date with the maximum concordance rate is “2020/5/22”. For example, the calculating function 114 determines the display candidate period “2020/5/22 to 2020/7/10”, which starts from the date “2020/5/22” when the concordance rate is the maximum and ends at the current date “2020/7/10”, to be the display period. That is, the calculating function 114 adjusts the display period from “2020/6/19” to “2020/7/10” to “2020/5/22” to “2020/7/10” so that the display period starts from the date “2020/5/22” when the concordance rate is maximum.

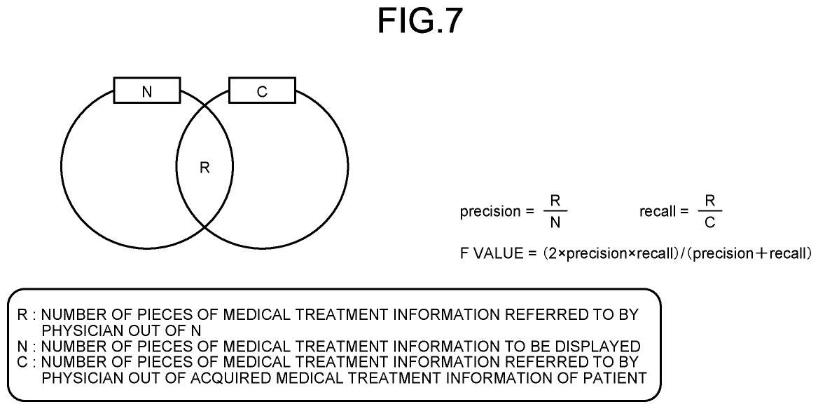

FIG. 7 is a diagram for describing a calculation method for the concordance rate. The calculating function 114 calculates the concordance rate by calculating the precision and the recall from the past reference determinations.

The precision corresponds to, for example, an index expressing how much medical treatment information referred to by the physician is included in the acquired medical treatment information of the patient. For example, the precision is the ratio of the value “R”, which is the number of pieces of medical treatment information referred to in the medical treatment information included in the display candidate period, to the value “N”, which is the number of pieces of medical treatment information included in the display candidate period in the acquired medical treatment information, and is calculated by Precision=R/N. For example, the value “R” represents the number of reference determinations “◯” as the number of pieces of medical treatment information referred to by the physician out of the value “N”. The value “N” is an example of “first value” and the value “R” is an example of “second value”.

The recall corresponds to, for example, an index expressing how comprehensively the medical treatment information referred to by the physician is displayed. For example, the recall is the ratio of the value “R” to the value “C,” which is the number of pieces of medical treatment information referred to in the acquired medical treatment information, and is calculated by Recall=R/C. For example, the value “C” represents the number of reference determinations “◯” in the acquired medical treatment information. The value “C” is an example of “third value”.

To increase the recall (Recall=R/C), R may be increased by extending the display candidate period, i.e., by increasing N. In this case, however, the precision (precision=R/N) will decrease. Thus, since there is a trade-off relation between the recall and the precision, the calculating function 114 calculates the F value, which is the harmonic mean of the precision and the recall, as the concordance rate. The F value is calculated by F=(2×precision×Recall)/(precision+Recall)=2R/(N+C).

Here, the calculating function 114 calculates the F value in a plurality of display candidate periods and determines the display period on the basis of the display candidate period with the maximum F value. In the example in FIG. 6, the calculating function 114 determines the display candidate period “2020/5/22 to 2020/7/10” with the maximum F value to be the display period.

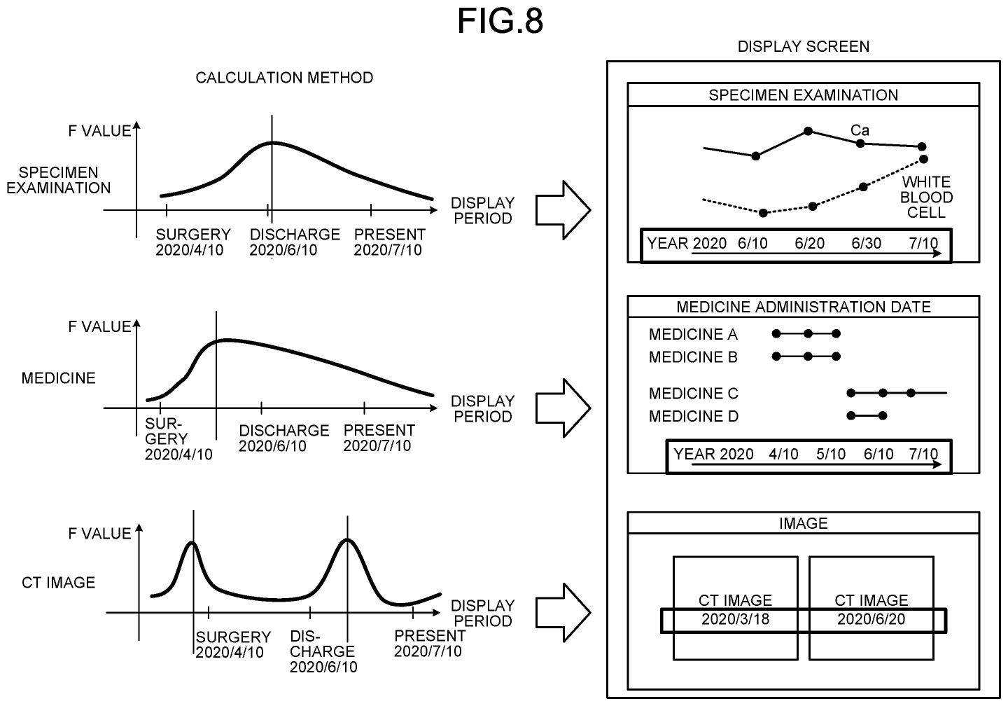

For example, the calculating function 114 calculates, by item, the F value on the basis of the operation information. In the example in FIG. 8, the calculating function 114 determines the display candidate period “2020/6/10 to 2020/7/10” with the maximum F value in the medical treatment information “Ca (calcium)” and “white blood cell” in the item “specimen examination” to be the display period. In this example, the period immediately after the discharge from the hospital to the current date is determined to be the display period.

The calculating function 114 determines the display candidate period “2020/4/10 to 2020/7/10” including the date with the maximum F value in the medical treatment information “medicine A” to “medicine D” in the item “medicine administration date” to be the display period. In this example, the period from the surgery followed by the discharge from the hospital to the current date is determined to be the display period.

The calculating function 114 is also applicable to examinations using medical images, and determines the two dates “2020/3/18” and “2020/6/20” when the F value becomes local maximum (peak) in the item “CT image” to be the display period. In this example, the date before the surgery and the date after the discharge are determined to be the display period.

Thus, the calculating function 114 determines the appropriate display period that the physician may want to check for different types of medical treatment information.

Here, the controlling function 115 of the electronic medical record server 100 selects one or more items to be displayed on the terminal 10 of the physician. For example, the physician operating the terminal 10 may select the items, or the items referred to more frequently by the physician may be selected first. Alternatively, the selected items are obtained by input of information such as the target patient's disease into a learned model obtained by machine learning. For example, based on result data expressing the items that the physicians selected, items that were referred to very frequently by physicians are specified, and a learned model is generated by learning using the result data. The controlling function 115 specifies the items by input of information such as the target patient's disease into the learned model.

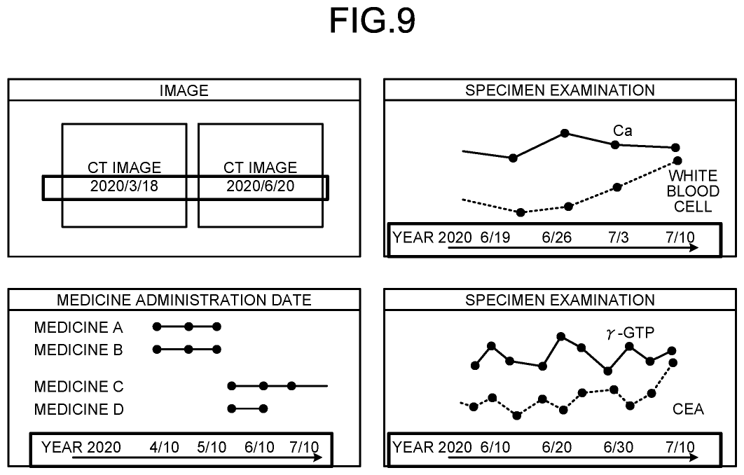

The controlling function 115 then causes the terminal 10 of the physician to display, by selected item, the medical treatment information for the display period determined by the calculating function 114. In the example in FIG. 9, the controlling function 115 causes the terminal 10 to display the medical treatment information for the display period “2020/6/19 to 2020/7/10” determined by the calculating function 114 as the medical treatment information “Ca” and “white blood cell” in the item “specimen examination”. The controlling function 115 causes the terminal 10 to display the medical treatment information for the display period “2020/6/10 to 2020/7/10” determined by the calculating function 114 as the medical treatment information “γ-Glutamyl TransPeptidase (γ-GTP)” and “carcinoembryonic antigen (CEA)” in the item “specimen examination”. The controlling function 115 causes the terminal 10 to display the medical treatment information for the display period “2020/4/10 to 2020/7/10” determined by the calculating function 114 as the medical treatment information “medicine A” to “medicine D” in the item “medicine administration date”. In examinations using medical images, the controlling function 115 causes the terminal 10 to display the CT images on the dates “2020/3/18” and “2020/6/20” determined by the calculating function 114 as the item “CT image”.

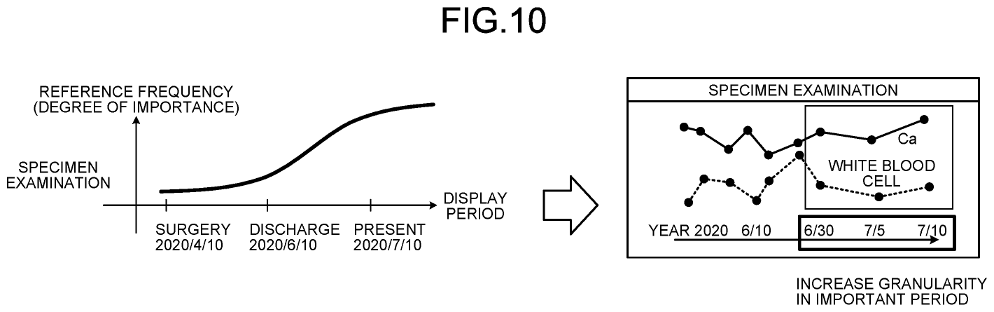

As a first modification, the calculating function 114 may determine the period with the most frequent reference to be an important period in the determined display period, and the controlling function 115 may perform display in a larger granularity in the important period in the display period determined by the calculating function 114. For example, the calculating function 114 determines the display candidate period “2020/6/10 to 2020/7/10” with the maximum F value in the medical treatment information “Ca” and “white blood cell” in the item “specimen examination” to be the display period. Here, the calculating function 114 determines the period in which the physician refers to the medical treatment information “Ca” and “white blood cell” frequently in the display period “2020/6/10 to 2020/7/10” determined by himself/herself to be the important period. For example, the frequency of reference exceeds the set frequency in the period “2020/6/30 to 2020/7/10” in the display period “2020/6/10 to 2020/7/10”. In this case, the calculating function 114 determines the period “2020/6/30 to 2020/7/10” to be the important period, and the controlling function 115, as illustrated in FIG. 10, causes the terminal 10 of the physician to perform display in the larger granularity in the important period “2020/6/30 to 2020/7/10” in the display period “2020/6/10 to 2020/7/10” so that the details in the important period “2020/6/30 to 2020/7/10” can be understood by the physician. For example, the controlling function 115 increases the size per day along the horizontal axis indicating the display period “2020/6/10 to 2020/7/10” for the important period “2020/6/30 to 2020/7/10” so that the physician can see the details of the important period on the terminal 10 of the physician.

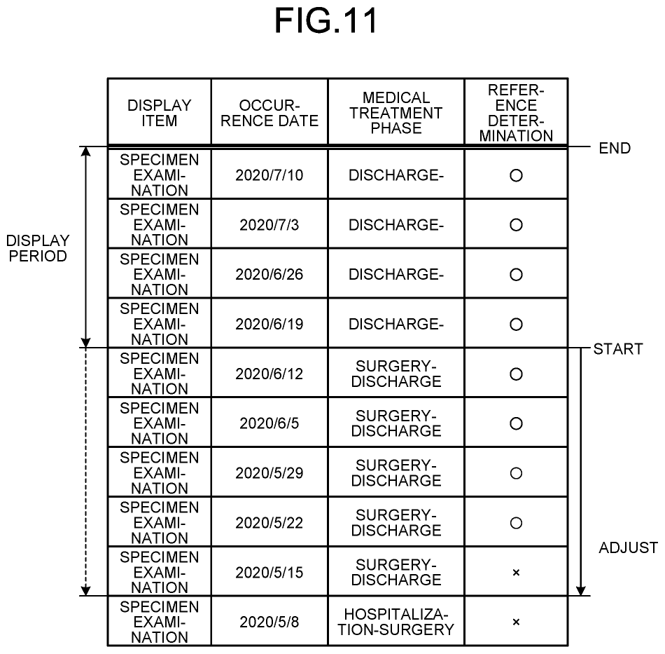

In the embodiment described above, the calculating function 114 determines the display candidate period with the maximum F value to be the display period, but in a second modification, the calculating function 114 may determine the display candidate period that includes the date with the maximum F value to be the display period. In the example in FIG. 11, the calculating function 114 determines the display candidate period “2020/6/19 to 2020/7/10” to be the display period, starting from the date “2020/6/19” when the F value is maximum and ending at the current date “2020/7/10” in the item “specimen examination”. Here, since the period “2020/6/19 to 2020/7/10” is the information after discharge of the target patient, if the information after the target patient's surgery is to be included, the display candidate period “2020/5/15 to 2020/7/10,” which includes the date “2020/6/19” with the maximum F value, is determined to be the display period. That is, the calculating function 114 determines “2020/5/15” to “2020/7/10” to be the display period by adjusting the start so as to include the date “2020/6/19” when the F value is maximum. In this case, the controlling function 115 causes the terminal 10 of the physician to display the medical treatment information for the display period “2020/5/15 to 2020/7/10” determined by the calculating function 114.

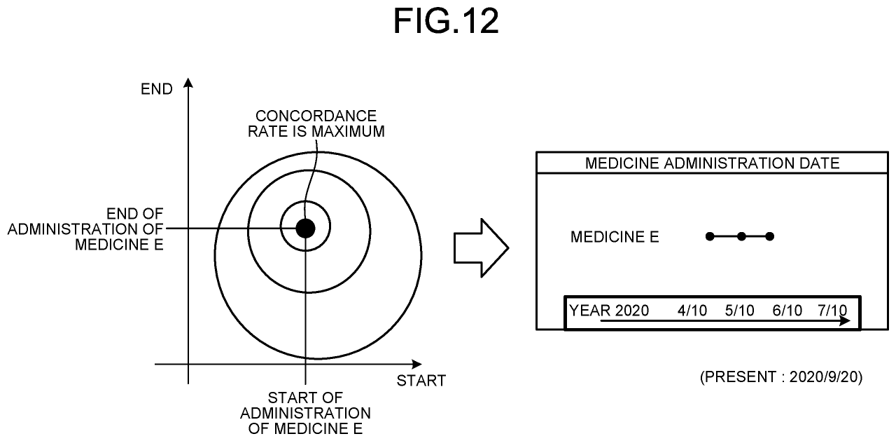

In the second modification, the calculating function 114 adjusts the start, but in a third modification, the calculating function 114 may adjust both the start and the end. In the example in FIG. 12, as for the calculating function 114, if the medical treatment information “medicine E” in the item “medicine administration date” includes the history of medicine administration of “medicine E” in the past, the period in which “the medicine E” was administrated includes the date when the F value is maximum. In this case, the calculating function 114 adjusts both the start and the end, calculates the F value in a plurality of periods from the start to the end, and determines the period with the maximum F value among the periods to be the display period. Thus, the calculating function 114 determines the past period including the date when the F value is maximum to be the display period. In this case, the controlling function 115 causes the terminal 10 of the physician to display the medical treatment information for the display period determined by the calculating function 114.

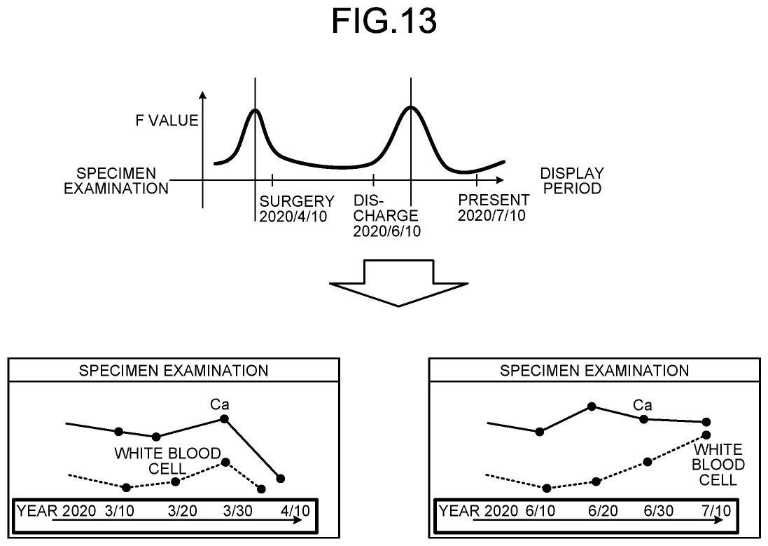

In the third modification, the calculating function 114 determines the past period including the date with the maximum F value to be the display period. In a fourth modification, the calculating function 114 determines the past period including the date with the local maximum (peak) F value and the period including the date with the local maximum F value and the current date to be the display period. In the example in FIG. 13, the calculating function 114 determines the display candidate periods “2020/3/10 to 2020/4/10” and “2020/6/10 to 2020/7/10” that include the date with the maximum F value in the medical treatment information “Ca” and “white blood cell” in the item “specimen examination” to be the display period. In this case, the controlling function 115 causes the terminal 10 of the physician to display the medical treatment information “Ca” and “white blood cell” for the display period “2020/3/10 to 2020/4/10” and the medical treatment information “Ca” and “white blood cell” for the display period “2020/6/10 to 2020/7/10”.

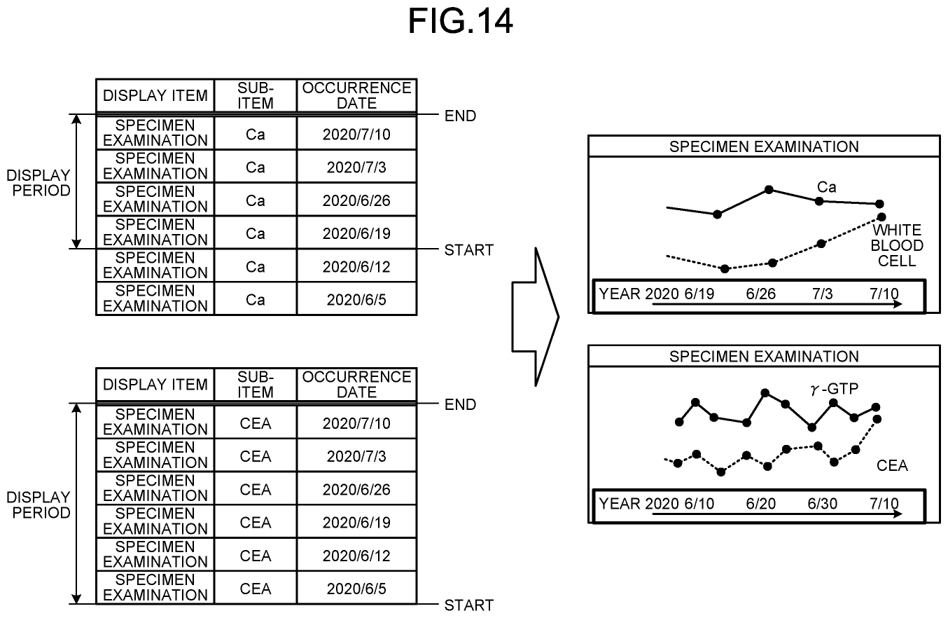

As a fifth modification, the calculating function 114 may determine the display period in accordance with the relevance of the sub-items. For example, as expressed in the left part of FIG. 14, the calculating function 114 determines the display candidate period “2020/6/19 to 2020/7/10” to be the display period, starting from the date “2020/6/19” with the maximum F value in the sub-items “Ca” and “white blood cell” as the medical treatment information in the item “specimen examination” and ending at the current date “2020/7/10”, and determines the display candidate period “2020/6/5 to 2020/7/10” to be the display period, starting from the date “2020/6/5” with the maximum F value in the sub-items “γ-GTP” and “CEA” and ending at the current date “2020/7/10”. Here, for example, if the relevance of the sub-items is high because the temporal variation of the sub-items “Ca” and “white blood cell” and the temporal variation of the sub-items “γ-GTP” and “CEA” are similar as the medical treatment information, for example, the calculating function 114 selects “2020/6/5 to 2020/7/10” with the maximum display period. In this case, the controlling function 115 causes the terminal 10 of the physician to display the medical treatment information “Ca”, “white blood cell”, “γ-GTP”, and “CEA” for the display period “2020/6/5 to 2020/7/10”.

On the other hand, if the relevance of the sub-items is low because the temporal variation of the sub-items “Ca” and “white blood cell” and the temporal variation of the sub-items “γ-GTP” and “CEA” are not similar as the medical treatment information, for example, the calculating function 114 does not select the display period. In this case, as illustrated in the right part of FIG. 14, the controlling function 115 causes the terminal 10 of the physician to separately display the medical treatment information “Ca” and “white blood cell” for the display period “2020/6/19 to 2020/7/10” and the medical treatment information “γ-GTP” and “CEA” for the display period “2020/6/5 to 2020/7/10”.

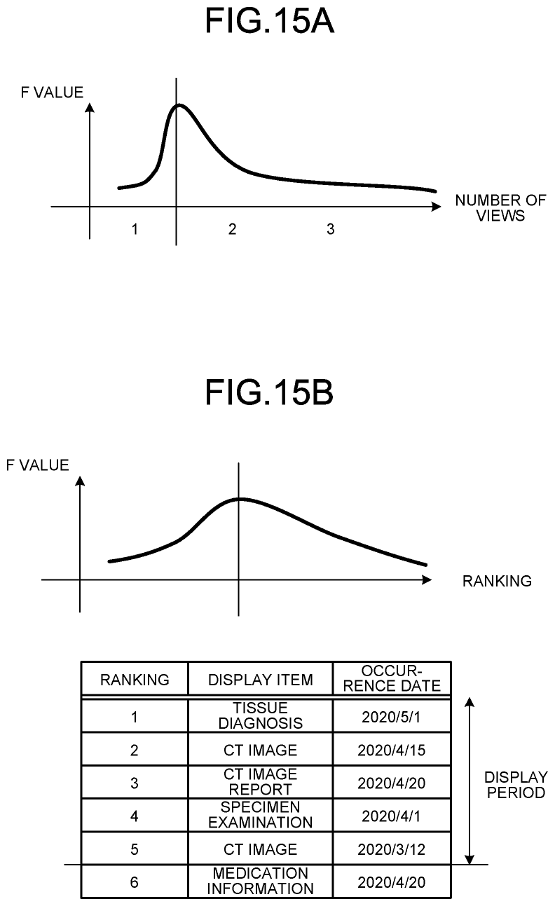

As a sixth modification, the calculating function 114 may determine the display period using the frequency of reference (views) as well as the length of period. For example, as illustrated in FIG. 15A, if the frequency of reference at which the F value is maximum is “1.5” times in the medical treatment information of the item “specimen examination”, the calculating function 114 determines the display candidate period that includes the date when the F value is maximum and the reference frequency is “1.5” times or more to be the display period. The calculating function 114 may determine the display period using machine learning. For example, as illustrated in FIG. 15B, ranking in order of how frequent the medical treatment information of the target patient is displayed in each item is determined in advance by machine learning. For example, in the case of the item “specimen examination” that is the fourth in the ranking of the maximum F value, the calculating function 114 determines the display candidate period that includes the date with the maximum F value, “2020/3/12,” in the fifth-ranked item “CT examination” to be the display period.

As described above, in the electronic medical record server 100 in the first embodiment, the acquiring function 113 acquires the physician's operation information for the medical treatment information of the target patient or the physician's operation information for the medical treatment information of the similar patient who is similar to the target patient. Based on the operation information, the calculating function 114 calculates the F value, which is the concordance rate between the period for which the medical treatment information of the target patient or the medical treatment information of the similar patient is referred to and the period for which the medical treatment information of the target patient is displayed, and the controlling function 115 performs display of the information about the F value. Specifically, the calculating function 114 determines the period starting from the date when the F value is maximum and ending at the current date to be the display period, and the controlling function 115 performs the display of the medical treatment information for the display period determined by the calculating function 114. Alternatively, the calculating function 114 determines the period including the date when the F value is maximum to be the display period, and the controlling function 115 performs the display of the medical treatment information for the display period determined by the calculating function 114. Thus, the electronic medical record server 100 according to the first embodiment can prevent the oversight of the medical treatment information that needs to be checked.

### Second Embodiment

In a second embodiment, the acquiring function 113 specifies the medical treatment scene of the target patient and acquires the physician's operation information for the medical treatment information of the target patient according to the specified medical treatment scene. For example, the medical treatment scene includes information about the target patient's disease. The information about the disease includes the name of the disease, the site of the disease, the treatment method, and the like. For example, the medical treatment scene includes a medical treatment phase, which is a period of time sectioned for each event. Examples of the medical treatment phase include a period from the initial consultation to hospitalization, a period from the hospitalization to a surgery, and a period from the surgery to discharge.

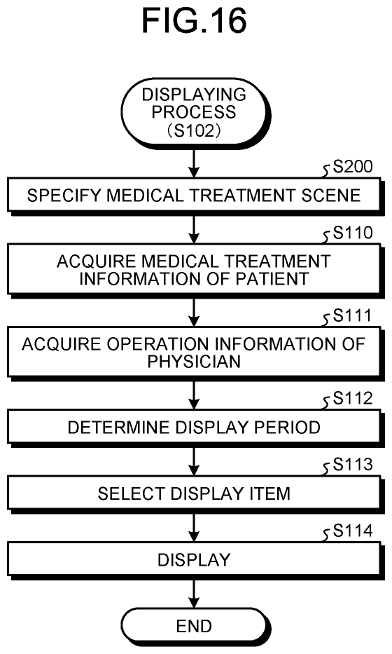

FIG. 16 is a flowchart expressing a procedure of a process by an electronic medical record server according to the second embodiment. In the second embodiment, at step S200 in FIG. 16, the acquiring function 113 specifies the medical treatment scene of the target patient by referring to the medical treatment information of the patient registered in the medical treatment information DB 121. After that, steps S110 to S114 in the first embodiment are performed. In this case, at steps S110 and S111, the acquiring function 113 acquires, by item, the patient's medical treatment information from the medical treatment information DB 121, according to the specified medical scene, and the physician's operation information for the patient's medical treatment information. At step S112, the calculating function 114 calculates the F value for the medical treatment information of the target patient on the basis of the operation information and determines the display period using the calculated F value. At steps S113 and S114, the controlling function 115 selects the items to be displayed on the terminal 10 of the physician, and causes the terminal 10 to display, by item, the medical treatment information for the determined display period.

In the second embodiment, the acquiring function 113 can acquire the physician's operation information (action data) according to the medical treatment scene. For example, the acquiring function 113 acquires the medical treatment information of the target patient at step S110. Furthermore, at step S110, the acquiring function 113 acquires the medical treatment information of a similar patient who was in the medical treatment scene similar to the medical treatment scene specified at step S200, and who was treated by the physician in charge of the target patient. Then, the acquiring function 113 acquires the operation information about the operation performed on the similar patient by the physician in charge of the target patient at step S111. The similar patient may be one or more patients. The calculating function 114 determines the display period for the target patient on the basis of the operation information of the operation performed on the similar patient at step S112. For example, the calculating function 114 determines the display period for the target patient by applying the period determined for the similar patient to the target patient. Thus, the calculating function 114 can determine the display period according to the preference of the physician in charge of the target patient.

Alternatively, the similar patient targeted in the above example may be the similar patient who was in the medical treatment scene similar to the medical treatment scene specified at step S200, and who was treated by the physician in charge of the target patient and a physician with the same attribute as that of the physician. Thus, the calculating function 114 can determine the display period according to the preferences of the group to which the physician in charge of the target patient belongs.

Alternatively, the similar patient targeted in the above example may be the similar patient who was in the medical treatment scene similar to the medical treatment scene specified at step S200 and treated by a physician of any attribute. Here, the attributes include young, experienced, and specialist physicians. For example, the calculating function 114 determines the display period of the experienced physician, so that the controlling function 115 can provide educational information to younger physicians about what period of time the experienced physicians refer to in making their diagnosis. In other words, by comparing the display period of a younger physician with that of an experienced physician, the younger physician can find out points to be improved. In addition, since the calculating function 114 determines the display period of the specialist physician, the controlling function 115 can provide the point of view that only the specialist physician can provide.

In the second embodiment, the medical department may be used as the attribute. For example, if the target patient has an urgent disease, the acquiring function 113 acquires the medical treatment information and the operation information for the similar patient with the urgent disease at step S111, so that the calculating function 114 can determine the display period suitable for the patient with the urgent disease at step S112. For example, a display period for displaying the most recent medical treatment information is determined for the target patient with the urgent disease. On the other hand, if the target patient has a chronic disease, the acquiring function 113 acquires the medical treatment information and the operation information for the similar patient with the chronic disease at step S111, so that the calculating function 114 can determine the display period suitable for the patient with the chronic disease at step S112. For example, for the target patient with the chronic disease, the long display period is determined for displaying the medical treatment information.

In addition, in the second embodiment, depending on the medical treatment scene, the operation information according to the patient's condition can be acquired. For example, if the target patient has a sudden symptom, the acquiring function 113 acquires the medical treatment information and the operation information for the similar patient who had the sudden symptom at step S111, so that the calculating function 114 can determine the display period suitable for the patient with the sudden symptom at step S112. For example, for the target patient with the sudden symptoms, the display period for displaying the most recent medical treatment information is determined.

In addition, in the second embodiment, the operation information in accordance with the patient's location can be acquired depending on the medical treatment scene. For example, if the target patient is an outpatient, the acquiring function 113 acquires the medical treatment information and the operation information for the similar patient during the outpatient treatment at step S111, so that the calculating function 114 can determine the display period suitable for the outpatient at step S112. For example, for outpatients, the long display period is determined for displaying the medical treatment information. On the other hand, if the target patient is hospitalized, the acquiring function 113 acquires the medical treatment information and the operation information for the similar patient in the medical treatment during the hospitalization at step S111, so that the calculating function 114 can determine the display period suitable for the hospitalized patient at step S112. For example, for the hospitalized patient, the display period for displaying the most recent medical treatment information is determined.

Thus, the electronic medical record server 100 according to the second embodiment can determine the display period in accordance with the medical treatment scene.

### Third Embodiment

A physician may refer to pieces of medical treatment information in combination. For example, a physician administers medicine to a target patient in consideration of the patient's symptoms, and checks the effectiveness and side effects of the medicine. In view of this, in a third embodiment, the calculating function 114 calculates the F value by combining the medical treatment information of the target patient acquired by item, and determines the display period using the calculated F value.

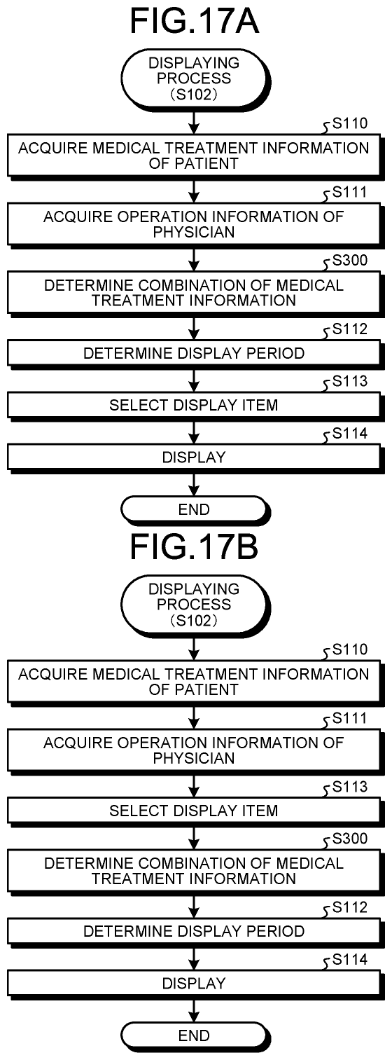

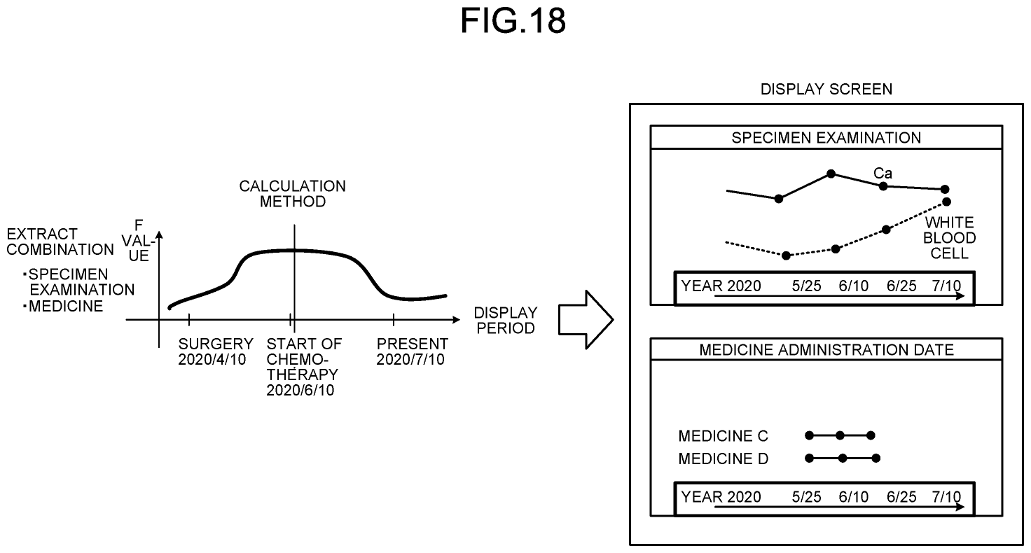

FIG. 17A is a flowchart expressing a procedure of a process by an electronic medical record server according to the third embodiment. In the third embodiment, after steps S110 and S111 in the first embodiment are performed, the calculating function 114 determines the combination of the medical treatment information of the target patient acquired by item, at step S300 in FIG. 17A. In the example in FIG. 18, the calculating function 114 determines the combination of the medical treatment information “Ca” and “white blood cell” in the item “specimen examination” and the medical treatment information “medicine C” and “medicine D” in the item “medicine administration date” on the basis of the start of chemotherapy at step S300 and determines the display candidate period “2020/5/25 to 2020/7/10” including the date “2020/6/10” when the chemotherapy is started to be the display period at step S112. After that, steps S112 to S114 in the first embodiment are performed.

Here, as illustrated in FIG. 17B, step S300 may be performed after steps S110, S111, and S113 in the first embodiment, followed by steps S112 and S114 in the first embodiment.

Thus, the electronic medical record server 100 according to the third embodiment can determine the display period considering the combination of the medical treatment information.

### Fourth Embodiment

A physician may want to check the past symptoms and treatment history of the target patient or similar patients whose diseases and other conditions are similar to those of the target patient. In view of this, in a fourth embodiment, by determining the display target period using the past action patterns, the calculating function 114 determines the display period in the determined display target period. In this case, the physician may determine the display target period, or the acquiring function 113 may determine the display target period. For example, the display target period is the period for which the medical treatment information was referred to very frequently in the past according to the click operations, eye tracking, the access history, or the like.

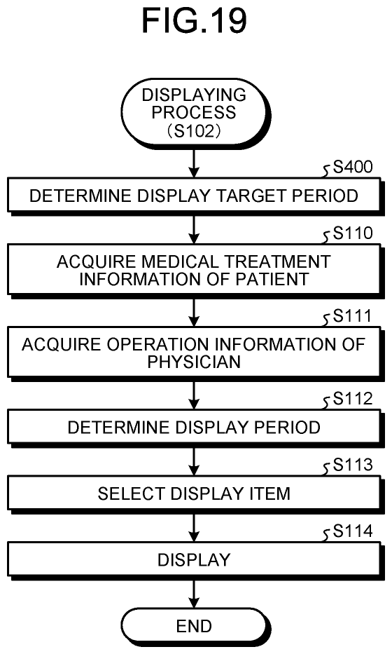

FIG. 19 is a flowchart expressing a procedure of a process by an electronic medical record server according to the fourth embodiment. In the fourth embodiment, for example, at step S400 in FIG. 19, the acquiring function 113 determines the display target period according to the click operations, eye tracking, the access history, or the like. After that, steps S110 to S114 in the first embodiment are performed. In this case, at steps S110 and S111, the acquiring function 113 acquires from the medical treatment information DB 121, the medical treatment information of the target patient by item in the determined display target period, and the physician's operation information for the medical treatment information of the target patient. At step S112, the calculating function 114 calculates the F value for the medical treatment information of the target patient on the basis of the operation information and determines the display period using the calculated F value. At steps S113 and S114, the controlling function 115 selects the items to be displayed on the terminal 10 of the physician, and causes the terminal 10 to display, by item, the medical treatment information for the determined display period.

Thus, the electronic medical record server 100 according to the fourth embodiment can determine the display period in consideration of the past symptoms and treatment history of the target patient or the similar patient.

Each component of each device illustrated in the first embodiment to the fourth embodiment is conceptual in terms of function, and does not necessarily have to be physically configured as illustrated in the drawings. In other words, the specific form of dispersion and integration of the devices is not limited to that illustrated in the figure, but can be configured by functionally or physically dispersing or integrating all or some of them in arbitrary units according to various loads and usage conditions. Furthermore, all or any part of each processing function performed by each device can be realized by a CPU and a computer program analyzed and executed by the CPU, or by hardware using wired logic.

The methods described in the first embodiment to the fourth embodiment can also be realized by executing a computer program prepared in advance on a computer such as a personal computer or workstation. This computer program can be distributed via the Internet or other networks. This computer program can also be recorded on a computer-readable non-transitory recording medium, such as a hard disk, flexible disk (FD), CD-ROM, MO, or DVD, and executed by being read out from the recording medium by a computer.

According to at least one of the embodiments described above, medical treatment information that needs to be checked can be prevented from being overlooked.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to:
acquire operation information of a medical worker for medical treatment information of a target patient or operation information of a medical worker for medical treatment information of a similar patient who is similar to the target patient;
calculate a concordance rate between a period for which the medical treatment information of the target patient or the medical treatment information of the similar patient is referred to, and a period for which the medical treatment information of the target patient is displayed, based on the operation information; and
perform display of information about the concordance rate.

2. The medical information processing apparatus according to claim 1, wherein the processing circuitry is configured to:
determine a period starting from a date when the concordance rate is maximum and ending at a current date to be a display period; and
perform display of the medical treatment information for the display period.

3. The medical information processing apparatus according to claim 1, wherein the processing circuitry is configured to:
determine a period including a date when the concordance rate is maximum to be a display period; and
perform display of the medical treatment information for the display period.

4. The medical information processing apparatus according to claim 1, wherein the processing circuitry is configured to:
calculate the concordance rate in a plurality of periods by adjusting both a start and an end and determine a period in which the concordance rate is maximum among the periods to be a display period; and
perform display of the medical treatment information for the display period.

5. The medical information processing apparatus according to claim 1, wherein the processing circuitry is configured to:
determine a plurality of periods including a date when the concordance rate becomes local maximum to be a display period; and
perform display of the medical treatment information for the display period.

6. The medical information processing apparatus according to claim 2, wherein the processing circuitry is configured to:
determine a period for which the medical treatment information of the target patient or the medical treatment information of the similar patient is frequently referred to in the determined display period to be an important period; and
perform display in a larger granularity in the important period in the display period.

7. The medical information processing apparatus according to claim 1, wherein the processing circuitry is configured to calculate, as the concordance rate, a harmonic mean of a ratio of a second value corresponding to the number of pieces of medical treatment information referred to in the medical treatment information included in a display candidate period in the acquired medical treatment information, to a first value corresponding to the number of pieces of medical treatment information included in the display candidate period, and a ratio of the second value to a third value corresponding to the number of pieces of medical treatment information referred to in the acquired medical treatment information.

8. The medical information processing apparatus according to claim 7, wherein the processing circuitry is configured to:
determine the display period, based on the display candidate period in which the concordance rate is maximum among a plurality of the concordance rates calculated in a plurality of the display candidate periods; and
perform display of the medical treatment information for the display period.

9. The medical information processing apparatus according to claim 1, wherein the processing circuitry is configured to:
acquire the medical treatment information of the target patient acquired by item, or the operation information for the medical treatment information of the similar patient acquired by the item;
calculate, by the item, the concordance rate, based on the operation information; and
perform display of information about the concordance rate by the item.

10. The medical information processing apparatus according to claim 9, wherein the processing circuitry is configured to select the item for which the information about the concordance rate is displayed.

11. The medical information processing apparatus according to claim 9, wherein the processing circuitry is configured to calculate the concordance rate by combining the medical treatment information of the target patient acquired by the item.

12. The medical information processing apparatus according to claim 1, wherein the processing circuitry is configured to acquire the operation information in accordance with a medical treatment scene.

13. The medical information processing apparatus according to claim 12, wherein the medical treatment scene includes information about a disease of the target patient.

14. The medical information processing apparatus according to claim 12, wherein the medical treatment scene includes a medical treatment phase sectioned for each event.

15. The medical information processing apparatus according to claim 1, wherein the operation information includes a time for which the medical worker refers to the medical treatment information as a content of operation by the medical worker.

16. The medical information processing apparatus according to claim 1, wherein the operation information includes a history when the medical worker accesses the medical treatment information as a content of operation by the medical worker.

17. The medical information processing apparatus according to claim 1, wherein the operation information is information operated by a plurality of the medical workers in hospital departments.

18. A medical information processing system comprising:
a plurality of terminals to be used by a plurality of medical workers; and
a medical information processing apparatus connected to the terminals through a network, wherein
the medical information processing apparatus includes a processing circuitry configured to:
acquire operation information of a medical worker for medical treatment information of a target patient or operation information of a medical worker for medical treatment information of a similar patient who is similar to the target patient;
calculate a concordance rate between a period for which the medical treatment information of the target patient or the medical treatment information of the similar patient is referred to, and a period for which the medical treatment information of the target patient is displayed, based on the operation information; and
perform display of information about the concordance rate.
